(19) United States
(12) Patent Application Publication
(10) Pub. No.: US 2009/0191260 A1
(43) Pub. Date: Jul. 30, 2009

(54) AEROSOL AND USE THEREOF

(75) Inventors: Knud Klingler, Engerwitzdorf (AT); Peter Steinrucke, Nürnberg (DE); Thorsten Bechert, Forchheim (DE)

Correspondence Address: PEARNE & GORDON LLP, 1801 EAST 9TH STREET, SUITE 1200, CLEVELAND, OH, 44114-3108, US

(73) Assignee: BIO-GATE AG, Nurnberg, DE (DE)

(21) Appl. No.: 12/280,404

(22) PCT Filed: Feb. 22, 2007
(86) PCT No.: PCT/EP2007/001528

§ 371 Date: Dec. 10, 2008

(30) Foreign Application Priority Data
Feb. 23, 2006 (DE) 10 2006 008 923.5
Apr. 12, 2006 (DE) 10 2006 017 679.0

(51) Int. Cl. A61K 9/127; A61K 9/12; A61K 9/14; A61K 33/38; A61P 29/00; A61P 31/00

(52) U.S. Cl. 424/450; 424/43; 424/489; 424/618

(57) ABSTRACT

The invention relates to an aerosol, in which a phase containing a solid substance is dispersed into a gaseous dispersant. The aim of the invention is to achieve a particularly effective anti-inflammatory action for the treatment of respiratory diseases. According to the invention, a depot is contained in the dispersed phase, said depot releasing silver ions when it comes into contact with water.

The invention relates to an aerosol according to the preamble of claim 1 and to uses of the aerosol and of a depot that releases silver ions when it comes into contact with water.

According to the prior art, it is generally known, for the treatment of respiratory diseases, to inhale anti-inflammatory and/or mucolytic substances in the form of an aerosol. To do so, the substances are usually added to hot water and inhaled. Generally, the symptoms are already alleviated during the inhalation. However, the anti-inflammatory action in particular of the inhaled substances is not especially long-lasting.

The object of the invention is to eliminate the disadvantages of the prior art. In particular, an aerosol and uses are proposed that permit treatment of inflammatory or infectious respiratory diseases, or inflammatory diseases of the organ of hearing, with improved efficacy.

This object is achieved by the features of claims 1, 15 and 16. Advantageous embodiments of the invention will become evident from the features of claims 2 to 14 and 17 to 30.

In an aerosol according to the invention, a depot that releases silver ions when it comes into contact with water is contained in the dispersed phase. A particularly long-lasting antimicrobial action can be achieved with the proposed aerosol. The aerosol is suitable in particular for the treatment of bronchitis, in particular chronic obstructive bronchitis, cystic fibrosis, inflammations of the lungs, and non-specific inflammations of the respiratory system with secondary effects. In addition, the proposed aerosol is also suitable for the treatment of inflammations of the organ of hearing, in particular for the treatment of acute and/or chronic inflammation of the middle ear or the like.

Within the meaning of the present invention “water” is also understood as aqueous solutions, aqueous body fluids, air moisture, and the like. A “depot” within the meaning of the present invention is an agent that provides a sustained release of silver ions when it comes into contact with water and that therefore has a sustained antimicrobial and thus anti-inflammatory action. A “depot” within the meaning of the present invention is an agent which, at room temperature, delivers silver ions at an antimicrobial concentration for more than one day, preferably for more than one week, particularly preferably for more than one month.

According to an advantageous embodiment of the invention, the depot contains aggregates formed from silver. The aggregates are advantageously formed from interconnected primary particles with a mean particle size of between 10 nm and 100 nm, preferably of 15 to 40 nm. The aggregates formed from the connected primary particles advantageously have a highly porous framework structure with a porosity of up to 95%, preferably 85 to 95%. Highly porous aggregates of this kind have a large inner surface area. The inner surface area can be from 3 to 6 m²/g, for example. Upon contact with water, silver ions detach from the surface of the aggregates in a therapeutically active concentration. The concentration is in particular such that undesirable cytotoxic side effects are avoided. In this connection, reference is furthermore made to the disclosure of WO 02/17984 A1.

According to another particularly advantageous embodiment, the aggregates have a mean particle size in the range of 1 to 10 μm, preferably 2 to 8 μm. It has been found that aggregates specifically with a mean particle size in the range of 2 to 8 μm can be inhaled particularly effectively.

It has been found that, in contrast to smaller particles, aggregates specifically with a particle size of greater than 2 μm can no longer be effectively identified by lung macrophages. Consequently, the aggregates according to the invention with a particle size of greater than 2 μm and preferably of less than 8 μm remain in the lungs for a long period of time and are there able to deploy a sustained antimicrobial action.

The aggregates advantageously have a spherical shape. This ensures that they do not become caught in the tissue and allows them in particular to be transported onwards, in particular in the area of the respiratory tract. Besides this, spherical aggregates provide a particularly uniform rate of release of silver ions. Their efficacy is easy to predict and, therefore, to adjust.

The silver used to produce the primary particles is particularly pure and advantageously contains only unavoidable impurities or is formed from a silver alloy with at least 80% by weight, preferably more than 99% by weight, of silver. Such silver has already proven effective in other areas for the treatment of inflammations. In this connection, reference is made, for example, to Br. J. Dermatl. (2005), 152(6), pages 1235 to 1242.

According to another embodiment, the aggregates are infiltrated with a therapeutically active substance. The substance is preferably an antimicrobial and/or vasodilating substance. The antimicrobial substances can be antibiotics or antiviral agents. Medicaments with a mucolytic action can also be readily infiltrated within the porous structure of the aggregates.

To this extent, therefore, the highly porous aggregates can also be used as carriers for transporting therapeutically active substances to the area of the airways. It is therefore possible to achieve a therapeutically excellent combination of fast-acting medicaments, infiltrated in the porous framework structure, with the depot action afforded by the aggregates themselves by release of silver ions.

According to a particularly advantageous embodiment, the depot is encased, preferably completely, by the liquid or the solid phase. The depot can be encased with a solid or a liquid phase after the silver aggregates have been infiltrated with a therapeutically active substance. The phase used for the encapsulation is chosen so as to facilitate the uptake and the transport of the depot, particularly in the respiratory organs. Moreover, the encapsulation can help prolong the dwell time in the alveolar area.

The dispersed phase, in particular the depot encased by the liquid or the solid phase, advantageously has a particle or droplet diameter of less than 10 μm and more than 100 nm. The mean particle or droplet diameters are advantageously in the range of 1 to 10 μm, preferably in the range of 5 μm. A dispersed phase with the aforementioned particle diameters can be breathed into the bronchi and alveoli.

The solid phase is advantageously formed from one of the following substances: liposome, vesicle-like structures or the like. Suitable liposomes are described in the following documents: Antimicrob. Agents Chemother., 38(5), pages 1090 to 1095 (1994); Antimicrob. Agents Chemother., 39(9), pages 2104 to 2111 (1995) and Anesthesiology, (83)2, pages 277 to 284 (1995). The disclosed content of these publications is hereby incorporated in the present document. The proposed solid phases permit an improved uptake of the depot, particularly in the area of inflamed and/or infected tissue.

According to another advantageous embodiment of the invention, the solid phase is a carbohydrate or a derivative thereof, a cyclodextrin, a protein, a biocompatible polymer, e.g. a dendrimer, or lactose, preferably α-lactose monohydrate. In this way, the aggregates according to the invention can be inhaled by powder inhalation. To this extent, the aggregates according to the invention are therefore present in the form of a powder whose mean particle size is in the range of 1 to 10 μm. Such powders are also referred to as micronized powders.

According to another embodiment, however, it is also possible for the aggregates according to the invention to be contained in a suspension for application. The suspension is of such a type that the aggregates do not precipitate. For this purpose, a highly viscous liquid is used in the suspension. The suspension can be applied by means of a suitable inhaler device. The aggregates according to the invention are released, for example in the area of a nozzle, by addition of an agent that reduces the viscosity. Such suspensions and application methods are known in the prior art. Reference is made, for example, to Drug Delivery 2004, September-October, 11(5): 295-300. It is also possible to keep the aggregates in the suspension by thixotropic effects.

Finally, it has proven advantageous for the depot to be contained in a concentration of 0.005 to 1.0% by weight, preferably 0.01 to 0.2% by weight, in the dispersed phase. With the proposed concentration, a long-lasting antimicrobial action can be achieved by the release of silver ions.

It is also possible for the depot according to the invention to be inhaled in powder form. In this case, the concentration of the phase, in particular of the silver aggregates, dispersed in air upon inhalation can also be higher.

According to another advantageous embodiment, the primary particles are interconnected via sinter necks. For this purpose, the primary particles can be produced in a conventional manner, for example by vacuum evaporation or the like. The primary particles are then subjected to a suitable heat treatment, such that sinter necks are formed to the desired extent and, therefore, aggregates are formed with the desired mean particle size distribution. Alternatively, it is also possible for the primary particles to be interconnected to form highly porous framework structures by using suitable compounds, for example thiofunctionalized polymers or crosslinkers.

Furthermore, the invention proposes that the aerosol according to the invention is used to produce a medicament. The proposed medicament can be used in particular for the treatment of inflammatory diseases, or diseases caused by microbial infection, in the area of the airways or the organ of hearing. In addition, the invention proposes the use of a depot, which releases silver ions when it comes into contact with water, to produce a medicament to treat inflammatory diseases, or diseases caused by microbial infection, in the area of the airways or the organ of hearing. The proposed medicaments are in each case administered in such a way that they are applied to, and thus brought into contact with, the internal mucous membranes in the area of the airways or the skin in the area of the organ of hearing. The medicament is preferably a medicament that is to be inhaled. However, the medicament can also be provided as a spray for application to the skin.

Within the meaning of the present invention, the term “airways” is to be understood generally as those organs and/or tissue areas that can be reached in particular by inhalation, rinsing or gargling. In addition to the lungs and bronchi, these also include, for example, the pharynx, nose or mouth, and the paranasal or maxillary sinuses. In the area of the pharynx, the proposed depot can be used in particular to treat diseases, for example, of the soft palate and tonsils, inflammation of the throat, tonsillitis, etc. In the area of the nose, it can be used particularly effectively to combat diseases of the nasal mucosa, e.g. colds, but in particular inflammation of the frontal and maxillary sinuses. In the mouth, it can be used to treat, among other things, inflammatory diseases of the gums and diseases in the area of the cheeks, palate and tonsils.

Diseases of the upper airways in particular are often initially caused by viruses. It has been found that silver ions in particular have an antiviral action and can therefore be used to effectively treat diseases of the upper airways even in the early stages.

Inflammation of the lungs is often caused by bacterial infections. By virtue of the excellent antimicrobial action of silver ions, such infections can be treated extremely efficiently using a depot that delivers silver ions. It has proven particularly advantageous that silver ions act against various types of bacteria and viruses. In order to achieve the efficacy according to the invention, expensive production of different specific pharmaceutical active substances in particular is therefore unnecessary.

For the advantageous embodiments of the depot, reference is made to the explanations given above.

To administer the depot, it can be taken up either in a liquid or in a preferably powdered solid phase. For the advantageous embodiment of the liquid, the solid phase and the concentration, reference is again made to the explanations given above.

The depot is advantageously inhaled in particular for the treatment of respiratory diseases. For this purpose, the depot, taken up in a liquid for example, can be for example sprayed or atomized using a suitable device. Similarly, the depot can be present in the form of an ointment, for example, and can be vaporized and inhaled using hot water.

It is likewise possible for a depot taken up in a liquid to be converted into an aerosol by means of an atomizer. For this purpose, a nozzle-type atomizer or an ultrasonic atomizer can be used, for example. In order in particular to loosen tough mucus present in the bronchi, the aerosol according to the invention can also be inhaled by means of jet inhalation. In this case, oscillations and pressure surges are induced in the inhaled aerosol stream by means of a compressor. In addition, the proposed depot can also be administered using metered aerosols. These involve in particular pharmaceutical products in which an aerosol with the depot is released from a container when a button is pressed. Finally, the depot can also be sprayed in the form of a dry powder and inhaled. In this case, metal particles forming the depot can be encased by suitable solid carrier substances, for example liposomes or the like.

Illustrative embodiments of the invention are explained in more detail below with reference to the drawings, in which:

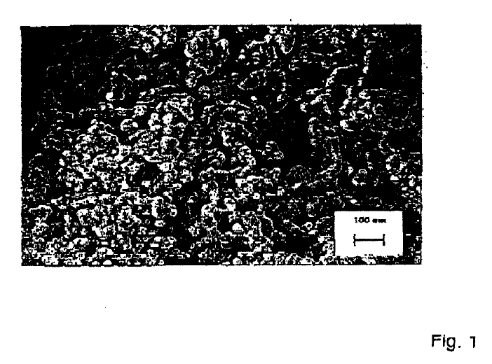

FIG. 1 shows a scanning electron microscope image of a silver aggregate;

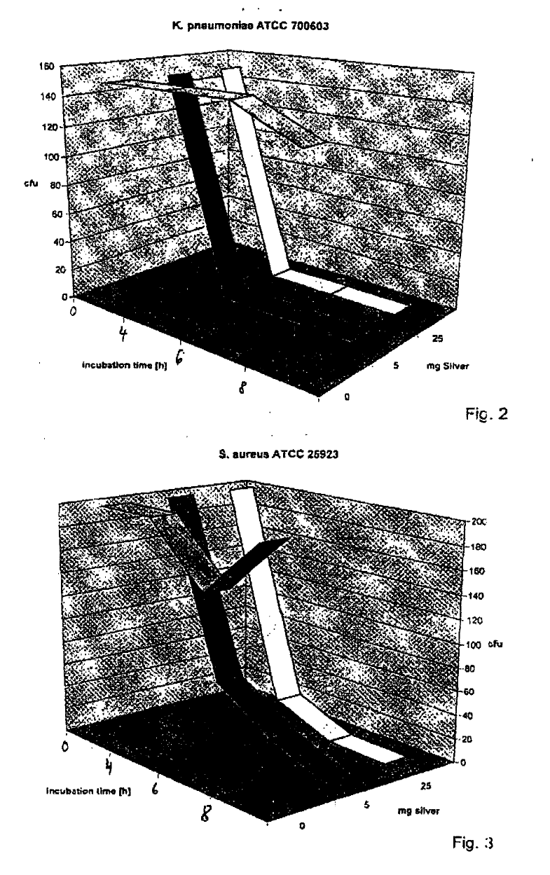

FIG. 2 shows the efficacy of the silver aggregates in respect of a Gram-negative microorganism; and

FIG. 3 shows the efficacy of the silver aggregates in respect of a Gram-positive microorganism.

FIG. 1 is a scanning electron microscope image of a silver aggregate according to the invention. The silver aggregate is composed mainly of spherical primary particles with a mean particle size of approximately 15 to 25 nm. The primary particles are largely interconnected via sinter necks. These are material bridges which are formed from silver and which, during the early phase of a sintering process, arise as a result of diffusion processes. Instead of the sinter necks, however, it is also possible for the primary particles to be interconnected by organic compounds, in particular crosslinkers, to form a highly porous framework. Such a highly porous framework is very suitable for the infiltration of therapeutically active substances. It can therefore be used as a chemically inert carrier for medicaments. The carrier has the therapeutically advantageous effect of permitting sustained release of silver ions in an antimicrobial concentration that is not cytotoxic. In the silver aggregates according to the invention, the primary particles are so firmly interconnected that the highly porous aggregate structure, when taken into the body, remains stable over a long period of time, i.e. at least one day, preferably longer than a month. The large inner surface area thus provided leads to slow and constant release of antimicrobial silver ions into the surrounding tissue. In this way, the frequency of a treatment can be greatly reduced. In particular, a treatment of respiratory diseases with the proposed aerosol places much less burden on the patient.

Using Klebsiella pneumoniae ATCC 700603 as an example, FIG. 2 shows the kinetics of destroying a Gram-negative microorganism. The quantities of silver aggregates indicated in FIG. 2 were added to a minimal medium (50 ml) in which the microorganisms remained substantially vital without addition of silver. The number of living microorganisms was determined after 0, 4, 6 and 8 hours for different concentrations of silver aggregates. Here, 100 cfu (colony forming units) in the view shown in FIG. 2 correspond to a microorganism count of 5000 microorganisms per ml.

As can be seen from FIG. 2, a concentration of just 0.01% by weight of silver aggregates, corresponding to 5 mg of silver aggregates in the batch, leads to complete destruction of the microorganisms within 6 hours.

FIG. 3 shows the efficacy of the silver aggregates in respect of a Gram-positive microorganism, here Staphylococcus aureus ATCC 25923. Here, the quantities of silver aggregates indicated in FIG. 3 were added to a minimal medium (50 ml) in which the microorganisms remained substantially vital without addition of silver. The number of living microorganisms was determined after 0, 4, 6 and 8 hours for different concentrations of silver aggregates. Here, 200 cfu in the view shown in FIG. 3 correspond to a microorganism count of 10 000 microorganisms per ml.

As can be seen from FIG. 3, a concentration of just 0.01% by weight of silver aggregates, corresponding to 5 mg of silver aggregates in the batch, leads to complete destruction of the microorganisms within 8 hours.

The silver aggregates used for inhalation can be present either as a dry powder or in a stable suspension. Such a suspension can be produced as follows:

A solution of 1 ml of Carbapol Aqua SF70 is added to 10 ml 150 mM of NaH₂PO₄. The pH is set to 6-7, preferably 6.8, by addition of 2 ml 0.5 M NaOH. 300 μl of a starting suspension containing the silver aggregates in a concentration of 40 mg/ml are pipetted in and mixed thoroughly using a vortex device. To produce the starting suspension containing the silver aggregates, a viscous liquid, for example glycerol (60 to 87.5%) is used.

The suspension thus produced was stable. After an incubation time of 48 hours, a voltammetric test of the silver ion content revealed a mean silver ion (Ag⁺) content of 8.5 μmol/L.

1. An aerosol, in which a phase containing a solid is dispersed in a gaseous dispersant, and a depot that releases silver ions when it comes into contact with water is contained in the dispersed phase, characterized in that the depot contains aggregates formed from silver that are formed from primary particles interconnected via sinter necks.

2. The aerosol as in claim 1, wherein the primary particles have a mean particle size of between 10 nm and 100 nm.

3. The aerosol as in claim 2, wherein the aggregates formed from the connected primary particles have a highly porous framework structure with a porosity of from about 85% to about 95%.

4. The aerosol as in claim 3, wherein the aggregates have a mean particle size in the range of 1 to 10 μm.

5. The aerosol as in claim 1, wherein the aggregates have a substantially spherical shape.

6. The aerosol as in claim 1, wherein the silver used to produce the primary particles contains unavoidable impurities or is formed from a silver alloy with at least 80% by weight of silver.

7. The aerosol as in claim 1, wherein the aggregates are infiltrated with a therapeutically active substance.

8. The aerosol as in claim 7, wherein the substance is an antimicrobial and/or vasodilating substance.

9. The aerosol as in claim 1, wherein the aggregates are encased by a liquid or the solid phase.

10. The aerosol as in claim 9, wherein the solid phase is a liposome, a carbohydrate or a derivative thereof, a cyclodextrin, a protein, a biocompatible polymer or lactose, preferably α-lactose monohydrate.

11. The aerosol as in claim 1, wherein the depot is contained in a concentration of 0.005 to 1.0% by weight in the dispersed phase.

12. A method of administering a medicament including silver ions comprising contacting airways or hearing organs with the dispersed phase of the aerosol of claim 1.

13. A method of treating inflammatory diseases, or diseases caused by microbial infection, in the area of the airways or the organ of hearing, comprising the steps of providing a depot that releases silver ions when the depot comes into contact with water, the depot containing aggregates of silver that are formed from primary particles interconnected via sinter necks, and contacting the airways or hearing organs with silver ions released by the depot contacting water.

14. The method of claim 13, wherein the primary particles have a mean particle size of between 10 nm and 100 nm.

15. The method of claim 13, wherein the aggregates formed from the connected primary particles have a highly porous framework structure with a porosity of from about 85% to about 95%.

16. The method of claim 13, wherein the aggregates have a mean particle size in the range of 1 to 10 μm.

17. The method of claim 13, wherein the aggregates have a substantially spherical shape.

18. The method of claim 13, wherein the silver used to produce the primary particles contains unavoidable impurities or is formed from a silver alloy with at least 80% by weight of silver.

19. The method of claim 13, wherein the aggregates are infiltrated with a therapeutically active substance.

20. The method of claim 19, wherein the substance is an antimicrobial and/or vasodilating substance.

21. The method of claim 13, wherein the aggregates are encased by a liquid or the solid phase.

22. The method of claim 21, wherein the solid phase is a liposome, a carbohydrate or a derivative thereof, a cyclodextrin, a protein, a biocompatible polymer or lactose, preferably α-lactose monohydrate.

23. The method of claim 13, wherein the depot is contained in a concentration of 0.005 to 1.0% by weight in the dispersed phase.

24. The method of claim 13, wherein the disease is an inflammatory disease, or a disease caused by microbial infection, affecting at least one of the following tissue areas and/or organs: pharynx, nose or mouth, paranasal or maxillary sinuses, soft palate, throat, vocal cords, gums, cheeks, tonsils, lungs, bronchi.

25. The method of claim 13, wherein the aggregates are highly porous and have a mean particle size in the range of 1 to 10 μm, and the primary particles have a mean particle size of between 10 nm and 100 nm.

26. The aerosol of claim 1, wherein the aggregates are highly porous and have a mean particle size in the range of 1 to 10 μm, and the primary particles have a mean particle size of between 10 nm and 100 nm.

27. The aerosol of claim 26, wherein the aggregates are infiltrated with a therapeutically active substance.

28. The aerosol of claim 26, wherein the aggregates are encased by a liquid or the solid phase.
